(19) United States
(12) Patent Application Publication
(10) Pub. No.: US 2023/0157577 A1
(43) Pub. Date: May 25, 2023

(54) NON-INVASIVE WEARABLE SENSOR DEVICE FOR DETECTING BIOMARKERS IN SECRETION

(71) Applicant: CHULALONGKORN UNIVERSITY, Bangkok (TH)

(72) Inventors: Nadnudda RODTHONGKUM, Bangkok (TH); Nadtinan PROMPHET, Bangkok (TH); Pranee RATTANAWALEEDIROJN, Bangkok (TH); Niphaphun SOATTHIYANON, Bangkok (TH); Krisana SIRALERTMUKUL, Bangkok (TH); Pranut POTIYARAJ, Bangkok (TH)

(73) Assignee: CHULALONGKORN UNIVERSITY, Bangkok (TH)

(21) Appl. No.: 17/905,587

(22) PCT Filed: May 27, 2020
(86) PCT No.: PCT/TH2020/000032

§ 371 Date: Sep. 2, 2022

(30) Foreign Application Priority Data
Mar. 6, 2020 (TH) 2001001314

(51) Int. Cl. A61B 5/103; A61B 5/279; A61B 5/263; A61B 5/256; A61B 5/00

(52) U.S. Cl. CPC A61B 5/1032; A61B 5/279; A61B 5/263; A61B 5/256; A61B 5/681; A61B 5/4266

(57) ABSTRACT

A non-invasive wearable sensor device for detecting biomarkers in secretion according to this invention comprises a colorimetric sensor (1), an electrochemical sensor (2), an electrochemical detector and processor (3) and a housing (4). The housing (4) is formed such that allows the colorimetric sensor (1) and electrochemical sensor (2) to contact with the secretion directly and continuously during wearing of the sensor device. This sensor device provides high performance of secretion absorption and retention, leading to high sensitivity to detection of biomarkers using a trace level of secretion sample. This sensor device is developed for detecting biomarkers based on two techniques: the colorimetric sensor (1) which allows the user to interpret a result by comparing it with a standard col or chart, and the electrochemical sensor (2) which provides a digital readout result. This sensor device can be used or simultaneous detection of several biomarkers in the same secretion sample.

### TECHNICAL FIELD

This invention is in a field of material and chemical science relating to a non-invasive wearable sensor device for detecting biomarkers in secretion.

### BACKGROUND OF THE INVENTION

Nowadays, the innovation of wearable sensor device is widely used in the applications of health care and preliminary diagnosis in order to monitor patient's health condition and indicate the wearer's health abnormality. In particular, the wearable sensor device with a non-invasive design can detect the samples with a real-time and continuous analysis since it is created to be worn on the human body for health monitoring. Sweat is the most suitable sample for the wearable sensor device because it is secreted from the human skin which can be collected continuously and is less contaminated compared to other secretion samples, such as tear, saliva or urine, etc. In sweat, there are several biomarkers which can indicate the health condition and can be used for medical diagnosis, for example, glucose, lactate, urea, creatinine and uric acid.

Owing to the advantages mentioned above, the wearable sensor device has been developed to be more efficient to be used in a wide range of applications. In general, the principle of biomarker detection relies on the specific interaction with the bio-receptors, such as enzyme, antibody, nucleic acid and DNA, immobilized on the surface of the sensor. The reaction of the biological substances with the target biomarkers leads to physical and chemical changes, such as electron flow, oxygen generation, change in moisture, heat and color, which can be detected using different techniques.

The detection techniques commonly integrated with the wearable sensor device for determination of biomarkers in sweat are the colorimetric and electrochemical techniques. From previous studies, the sensor was provided on flexible polymer substrates, which is grooved as small channels called microfluidic channels, so the secretion sample can flow through the channels and reach the sensor.

Rogers et al. (US 2018/0064377 A1) developed a colorimetric wearable sensor device by using flexible polymer substrates to detect the sweat rate, pH and concentration of chloride, glucose and lactate in sweat.

Bandodkar et al. (Science Advances, 5, 1-15, 2019) developed a wearable sensor device for determination of glucose, lactate, chloride ion, pH and sweat rate by using the colorimetric and electrochemical techniques and the volumetric analysis.

Javey et al. (US 2018/0263539 A1) developed a wearable sensor device for detecting glucose, lactate, sodium ion and potassium ion in sweat by using electrochemical detection together with monitoring human body's temperature. The sensor device was developed by installing the sensor, detector and processor on flexible polymer substrates, and using microfluidic pattern as a detection system, therefore leading to the use of smaller amount of reagents and samples. However, this system must be delicately prepared because of its complicated pattern. Therefore, this complexity in the fabrication process results in a high production cost.

Additionally, Jia et al. (Analytical Chemistry, 85, 14, 6553-6560, 2013) designed a tattoo-based wearable sensor which has a capability to monitor lactate in sweat during exercise.

Bandodkar et al. (Analyst, 138, 1, 123-128, 2013) developed a tattoo-based sensor for glucose determination in order to diagnose diabetes. These mentioned studies utilize the electrochemical technique by installing the wearable sensors on polymer substrates. By using such substrates, the ventilation rate of human body is apparently decreased, causing dampness and irritation on the skin.

The development of sensors using textiles, especially thread, as a base material is an alternative approach for creating simple, low-cost wearable sensor devices owing to the self-microfluidic property and the small size of the thread compared to other base materials which help to reduce the amount of enzymatic assay and simplify the fabrication and design process of the flow channel, therefore leading to cost reduction. Previously, several researchers developed a textile-based colorimetric sensor as following examples.

Xiao et al. (Cellulose, 26, 4553-4562, 2019) developed a colorimetric wearable sensor based on a combination of thread and filter paper and sewed on cloth for detection of glucose in sweat. However, the filter paper is fragile and lacks robustness for wearers with high sweat rate.

Li et al. (Cellulose, 25, 4831-4840, 2018) developed a colorimetric sensor based on a thread for detection of glucose in urine using a knotted thread for colorimetric readout. This thread-based sensor is more durable than the paper-based sensor. Nonetheless, on the difficulty in controlling the knot size can lead to inconsistency and change in color and the uneven surface can cause difficulty in interpretation and error.

There are several studies on the development of electrochemical wearable sensors.

Wang et al. (US2019/0090809 A1) developed a wearable sensor using screen-printed carbon electrode on clothes to detect β-nicotiamide adenine dinucleotide (NADH), hydrogen peroxide (H₂O₂), potassium ferrocyanide, trinitrotoluene (TNT), dinitrotoluene (DNT) in liquid and gas phases using amperometry or potentiometry technique. Nevertheless, the fabrication of the screen-printed working electrode requires a large amount of carbon ink, and the carbon electrode can be severely damaged while stretching the clothes. In addition, in this study, there is no modification made to the electrode with a conductive material for enhancing the electrical conductivity; therefore, leading to a poor electron transfer reaction on the electrode surface. Furthermore, any highly specific biological substance was not used, so interferences in the sample can disturb the detection mechanism. Thus, this sensor has suboptimal performance compared to other studies, which improve the performance of the sensors by modifying or improving the property of electrodes with a conductive material and highly specific biological substances.

Liu et al. (Lab on a Chip, 16, 2093-2097, 2016) developed an embroidered electrochemical sensor by coating carbon ink on the thread and using it as working and counter electrodes. For reference electrode, the thread is coated by Ag/AgCl ink. Then, the working electrode is further coated by a specific enzyme to detect glucose and lactate in blood. In order to detect the analytes in the blood sample, blood drawing, which is an invasive procedure, unavoidably causes pain and damage to the sampling sites and some patients have limitations for blood drawing. Furthermore, the embroidered electrochemical sensor embroidered on bandages (Biosensors and Bioelectronics, 98, 189-194, 2017) was developed for detection of biomarkers in wounds, such as uric acid. This platform can be fabricated by coating carbon ink and Ag/AgCl ink on the threads, and then embroidering the bandages using the threads.

On the other hand, Liu et al. used merely carbon ink for electrode modification and used a specific enzyme for the analysis. There is no modification step using the conductive material, therefore leading to a poor electron transfer process which causes low sensitivity and inefficiency of the analyte detection. Therefore, the detection efficiency and sensitivity can be improved by electrode modifications.

### SUMMARY OF THE INVENTION

A non-invasive wearable sensor device for detecting biomarkers in secretion of this invention was developed by using a combination of the colorimetric and electrochemical techniques to obtain a highly accurate and precise analysis. The invention uses a textile as a base material of the sensors because it has an outstanding self-microfluidity which allows self-absorption of the secretion samples. Moreover, this sensor can be readily fabricated at low cost while providing high efficiency and durability for being worn on human bodies. Herein, the novel sensor has been developed by modifying colorimetric and electrochemical sensor with a liquid absorber for high efficiency sample absorption and retention. In addition, it can increase the probability of absorbing and contacting with the target biomarkers in the secretion sample and the specific enzyme can be immobilized on the sensor even better. Moreover, modifying the base material using the liquid absorber before coating it with a conductive material and optionally a mediator on the electrochemical sensor can enhance the electrochemical conductivity of the sensor.

This sensor is not only suitable for the analysis of small amount of samples and highly sensitive to detection of the low concentration samples, but it can also be used for a simultaneous determination of various biomarkers in the samples by using the colorimetric technique in combination with the electrochemical technique for confirming the results in more accurate and precise manner.

This invention relates to a non-invasive wearable sensor device for detecting biomarkers in secretion. The sensor device comprises a colorimetric sensor comprising a base material coated with a liquid absorber, a colorimetric reagent and enzyme specific for target biomarkers, wherein when the colorimetric sensor contacts with the secretion, the enzyme specific for target biomarkers together with the colorimetric reagent causing the color change which is proportional to concentrations of the target biomarkers, and the colorimetric sensor is installed on a substrate such that it can be attached to and detached from the sensor device. The sensor device also comprises an electrochemical sensor comprising three electrodes, namely, a reference electrode (RE), a working electrode (WE) and a counter electrode (CE) that are installed on a substrate such that they can be attached to and detached from the sensor device. The electrochemical sensor is connected to an electrochemical detector and processor, and an end of those three electrodes is coated with a conductive material. The electrochemical detector and processor work together with the electrochemical sensor, and comprises a microcontroller, a real-time clock module, a battery as a power source, a button, a display and electrochemical circuits. The electrochemical circuit comprises an operational amplifier, a current source controller, a current-to-voltage converter, a digital-to-analog converter, analog-to-digital converters and a resistor. All components of the electrochemical detector and processor are electrically connected. The sensor device further comprises a housing to which the colorimetric sensor, electrochemical sensor and electrochemical detector and processor are installed. The housing is formed such that allows the colorimetric sensor and electrochemical sensor to contact with the secretion directly and continuously during wearing of the sensor device.

An object of this invention is to develop a non-invasive wearable sensor device for detecting biomarkers in secretion. The sensor device of this invention can be developed by using the base material which is a textile due to its self-microfluidity and high wearing comfort properties. The sensor is highly sensitive to detection of a trace level of secretion samples, therefore highly efficient and useful for tracking user's health status and enable the users to preliminary evaluate and record health information by themselves which would provide advantages in medical diagnosis. This can also facilitate the doctor and reduce the cost of hospital visits of the user. Therefore, this invention can improve people's life quality and reduce the medical cost for the government.

### BRIEF DESCRIPTION OF THE DRAWINGS

FIG. 1 is a schematic plan showing (1a) a front view and (1b) a back view of the non-invasive wearable sensor device of this invention.

FIG. 2 is a schematic plan showing (2a) a front view and (2b) a side view of the colorimetric sensor of this invention.

FIG. 3 is a schematic plan showing the electrochemical sensor of this invention.

FIG. 4 is a diagram showing the components of the electrochemical detector and processor of this invention.

FIG. 5 is a schematic plan showing the non-invasive wearable sensor device being placed on the user's skin for detecting biomarkers in sweat.

FIG. 6 is a flow chart depicting the protocol for using the non-invasive wearable sensor device for detecting biomarkers in secretion.

FIG. 7 is a standard color chart for glucose detection.

FIG. 8 is a standard color chart for lactate detection.

### DETAILED DESCRIPTION

The present invention relates to a non-invasive wearable sensor device for detecting biomarkers in secretion which will be described by the following details with reference to the accompanying figures.

The sensor device of this invention comprising:

a colorimetric sensor (1) comprising a base material coated with a liquid absorber, a colorimetric reagent and enzyme specific for target biomarkers,

wherein when the colorimetric sensor (1) contacts with the secretion, the enzyme specific for target biomarkers together with the colorimetric reagent causing the color change which is proportional to concentrations of the target biomarkers, and the colorimetric sensor (1) is installed on a substrate (5) such that it can be attached to and detached from the sensor device;

an electrochemical sensor (2) comprising three electrodes, namely, a reference electrode (RE) (6), a working electrode (WE) (7) and a counter electrode (CE) (8) that are installed on a substrate (10) such that they can be attached to and detached from the sensor device, wherein the electrochemical sensor (2) is connected to an electrochemical detector and processor (3), and

an end of the three electrodes (9) is coated with a conductive material, and

the working electrode (7) comprises a base material which is coated with a conductive material, liquid absorber and enzyme specific for target biomarkers, and optionally a mediator, and

optionally, more than one colorimetric sensor (1) or electrochemical sensor (2) is installed on the sensor device in order to detect several biomarkers simultaneously, and when the secretion contacts with the electrochemical sensor (2), the enzyme specific for target biomarkers being on the working electrode (7) reacts with the target biomarkers causing a number of electrons on a surface of the working electrode (7) that are converted into current signals passing through the electrochemical detector and processor (3), the current signals being proportional to concentrations of the target biomarkers, and the electrochemical detector and processor (3) that works together with the electrochemical sensor (2) comprising:
a microcontroller (12) which serves to control a digital-to-analog converter (DAC) to operate the current source, read the voltage input from a feedback voltage measuring module, read the voltage from a current-to-voltage converter, send the measurable value to a display, monitor and control a working process, and then read a real-time clock signal;
a real-time clock module (13) which serves to generate a current clock signal, and provide the microcontroller (12) with said current clock signal;
a battery (14) as a power source;
a button (15) which is used to switch modes and start the operation;
a display (16) that shows the measured result in the measure mode and shows current clock data;
electrochemical circuits (11, 25) comprising:
   an operational amplifier (18) which measures differential voltage between the working electrode (7) and reference electrode (6),
   a current source controller (19) which measures differential voltage between its two inputs,
   a current-to-voltage converter (20) which converts a current input into a voltage,
   a digital-to-analog converter (21) which converts the digital signal from the microcontroller (12) into an analog signal to control the current source,
   analog-to-digital converters (22, 23) which convert the analog signal into the digital signal, which will be recognized by the microcontroller, and
   a resistor (24) which is used for converting current into voltage,

wherein all components of the electrochemical detector and processor (3) are electrically connected and installed on a substrate, and

a number of the electrochemical circuits (11, 25) installed in the electrochemical detector and processor (3) corresponds to a number of the electrochemical sensor (2) installed on the sensor device;

a housing (4) to which the colorimetric sensor (1), electrochemical sensor (2), and electrochemical detector and processor (3) are installed,

wherein the housing (4) is formed such that allows the colorimetric sensor (1) and electrochemical sensor (2) to contact with the secretion directly and continuously during wearing of the sensor device, and

the housing (4) is made of a material that is selected from a group consisting of textile, paper, polymer, metal, ceramic and a combination thereof.

In one embodiment, the base material is made of a textile which is natural fiber, synthetic fiber, conductive fiber or a combination thereof, and is in a form of fiber, thread, fabric or a combination thereof.

The base material may be made of paper, polymer, metal, ceramic or a combination thereof.

The base material for the colorimetric sensor (1) and electrochemical sensor (2) can be made of the same or different material.

In a preferred embodiment, the mediator is selected from a group consisting of metal hexacyanoferrate, Prussian blue, cobalt hexacyanoferrate, cobalt phthalocyanine (CoPc), tetracyanoquinodimethane (TCNQ), potassium ferricyanide, ferrocene and its derivatives and a combination thereof.

The mediator has a concentration in a range of 0.001-10% by weight of the base material.

The liquid absorber is selected from a group consisting of positive ion, negative ion, carbon nanomaterial which is graphene or its derivatives, carbon nanotube, cationic or anionic polymer which is chitosan or its derivatives, cellulose or its derivatives, alginate or its derivatives, pullulan or its derivatives and a combination thereof.

The liquid absorber coated on the colorimetric sensor (1) and electrochemical sensor (2) has a concentration of in a range of 0.001-10% by weight of the base material.

The liquid absorber coated on the colorimetric sensor (1) and electrochemical sensor (2) can be the same or different material and comprise one or more types of material.

The colorimetric reagent is selected from a group consisting of aniline derivatives, i.e. N-ethyl-N-(3-sulfopropyl)-3-methoxyaniline, sodium salt, monohydrate (ADPS), N-ethyl-N-(3-sulfopropyl) aniline, sodium salt (ALPS), N-ethyl-N-(2-hydroxy-3-sulfopropyl)-3,5-dimethoxyaniline, sodium salt (DAOS), N-(2-hydroxy-3-sulfopropyl)-3,5-dimethoxyaniline, sodium salt (HDAOS), N,N-bis(4-sulfobutyl)-3,5-dimethylaniline, disodium salt (MADB), N-ethyl-N-(2-hydroxy-3-sulfopropyl)-3,5-dimethylaniline, sodium salt, monohydrate (MAOS), N,N-bis(4-sulfobutyl)-3-methylaniline, disodium salt (TODB), N-ethyl-N-(2-hydroxy sulfopropyl)-3-methylaniline, sodium salt, dihydrate (TOOS), N-ethyl-N-(3-sulfopropyl)-3-methylaniline, sodium salt, monohydrate (TOPS); benzidine derivatives, i.e. 3,3′-,5,5′-tetramethylbenzidine (TMBZ), 3,3′-,5,5′-tetramethylbenzidine, dihydrochloride, dihydrate, (TMB-HCl), 3,3-diaminobenzidine, tetrahydrochloride (DAB), 4-aminoantipyrine, potassium iodide; azo dyes; triphenylmethane dyes; fluorescent dyes; acridine dyes; miscellaneous dyes; anthraquinone dyes; sulfonephthalein dyes; benzein dyes; xanthene dyes; phthalein dyes; thiazole dyes; coumarin dyes; chalcone dyes; nitro dyes; heterocyclic dyes; polymethine dyes; flavone dyes; indigoid dyes; naphthalene dyes; azine dyes; oxazine dyes; hydrazide dyes; quinoline dyes; styryl dyes; oxazone dyes, i.e. bromocresol green, bromophenol red, methyl orange, methyl red, phenolphthalein, thymol blue, litmus, phenol red and a combination thereof.

The colorimetric reagent has a concentration in a range of 0.0001-10% by weight of the base material.

The enzyme specific for target biomarkers is selected from a group consisting of oxidase enzymes, i.e. glucose oxidase, horseradish peroxidase, lactate oxidase, cholesterol oxidase, creatinine amidohydrolase, urease and a combination thereof.

The enzyme specific for target biomarkers coated on the colorimetric sensor (1) and electrochemical sensor (2) has a concentration in a range of 0.01-1,000 units per gram of the base material.

The enzyme specific for target biomarkers coated on the colorimetric sensor (1) and electrochemical sensor (2) can be the same or different enzyme.

The reference electrode (6) is an ink or electrode which comprises carbon or Ag/AgCl as a main component.

The counter electrode (8) is an ink or electrode which comprises carbon, Ag/AgCl or platinum (Pt) as a main component.

The conductive material is selected from a group consisting of carbon-based nanomaterials, i.e. graphene or its derivatives, carbon nanotubes; metal-based nanoparticles, i.e. gold, silver, platinum, nickel, copper; conductive polymer, i.e. polyaniline, polypyrrole, poly(3,4-ethylenedioxy thiophene):polystyrene sulfonate; conductive ink or adhesive, i.e. Ag/AgCl ink, carbon ink; conductive tape, i.e. silver tape, copper tape and a combination thereof.

The conductive material coated on the working electrode (7) and the end of the three electrodes (9) has a concentration in a range of 1-1000% by weight of the base material.

The conductive material coated on the working electrode (7) and the end of the three electrodes (9) can be the same or different material.

The substrate of the colorimetric sensor (1), electrochemical sensor (2) and electrochemical detector and processor (3) is selected from a group consisting of textile, paper, polymer, metal, ceramic and a combination thereof.

The substrate of the colorimetric sensor (1), electrochemical sensor (2) and electrochemical detector and processor (3) can be the same or different material.

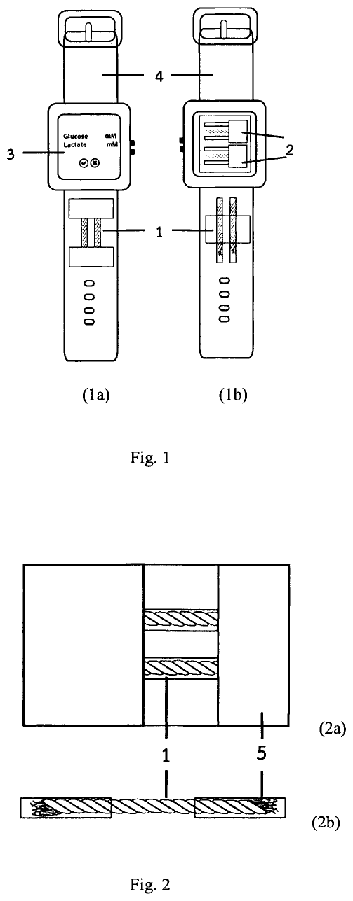

Referring to FIG. 1, the non-invasive wearable sensor device is fabricated for detection of biomarkers, such as glucose, lactate or urea in secretion, such as sweat, saliva, urine and tear. This wearable sensor device can be used for measuring the level of biomarkers by using the colorimetric and electrochemical techniques. To evaluate the result from the colorimetric experiment, an interpretation can be proceeded by comparing the color shade appearing on the sensor with the standard color chart. On the other hand, the result of the electrochemical technique can be interpreted by reading the result on the display of the device. Also, this invention can be used for simultaneous determination of several biomarkers. As described above, the wearable sensor device of this invention comprises four main parts: the colorimetric sensor (1), the electrochemical sensor (2), the electrochemical detector and processor (3) and the housing (4).

Referring to FIG. 2, the colorimetric sensor (1) comprises the base material coated with the liquid absorber, the colorimetric reagent and enzyme specific for target biomarkers. The liquid absorber on the colorimetric sensor (1) plays an important role in enhancing the secretion sample absorption from the area which the base material contacts with secretion and facilitating the immobilization of enzyme, leading to high efficiency in small sample detection. As soon as the secretion sample contacts with the colorimetric sensor (1), the immobilized enzyme will work together with the colorimetric agent, causing the change of color of the working area. The color intensity of the working area is directly proportional to the concentration of the biomarkers in the sample. The colorimetric sensor (1) is installed on the substrate (5) for ease of attachment and detachment of the colorimetric sensor (1) to and from the sensor device. The number of colorimetric sensor on the sensor device can be more than one.

An exemplary embodiment of the colorimetric sensor (1) according to this invention is shown in FIG. 2, the colorimetric sensor (1) comprises two threads for detecting two biomarkers, which are lactate and glucose. The upper thread is for lactate detection, while the bottom thread is for glucose detection.

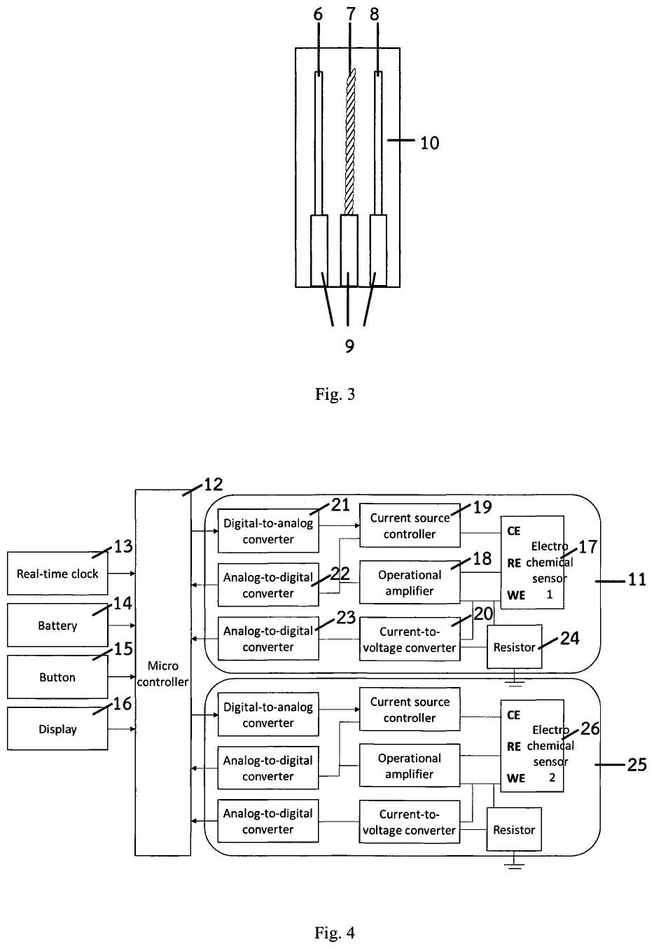

An exemplary embodiment of the electrochemical sensor (2) is shown in FIG. 3. The electrochemical sensor (2) comprises three electrodes: the reference electrode (6), working electrode (7) and counter electrode (8). These three electrodes are installed on the substrate (10) for ease of attachment and detachment of the electrochemical sensor (2) to and from the sensor device. For this electrochemical sensor (2) is connected to the electrochemical detector and processor (3), it should possess a good electrical conductivity. Therefore, the end of those three electrodes (9) must be coated with the conductive material. The working electrode (7) is fabricated from the base material which is coated with the conductive material, liquid absorber and enzyme specific for target biomarkers, and optionally mediator. When the secretion excreted from the body contacts with the electrochemical sensor (2), the immobilized enzyme on the working electrode (7) will specifically react with the target biomarkers, causing an electron flow in the system. The number of electrons on the surface of the working electrode (7) are converted into a current signal flowing through the electrochemical detector and processor (3). The current signal is directly proportional to the concentration of target biomarkers. The number of electrochemical sensor (2) equipped on the sensor device can be more than one.

As shown in FIG. 4, the electrochemical detector and processor (3) is an important component that works together with the electrochemical sensor (2). One electrochemical detector and processor (3) (for detecting one biomarker) comprises the following components:
the microcontroller (12) which serves to control a digital-to-analog converter (DAC) to operate the current source, read the voltage input from a feedback voltage measuring module, read the voltage from a current-to-voltage converter, and read a real-time clock signal;
the real-time clock module (13) which serves to generate a current clock signal, and provide the microcontroller with said current clock signal;
the battery (14) as a power source;
the button (15) which is used to switch modes and start the operation;
the display (16) that shows the measured result in the measure mode and shows current clock data;
the electrochemical circuits (11, 25) comprising:
   the operational amplifier (18) which measures differential voltage between the working electrode (7) and reference electrode (6),
   the current source controller (19) which measures differential voltage between its two inputs,
   the current-to-voltage converter (20) which converts a current input into a voltage,
   the digital-to-analog converter (21) which converts the digital signal from the microcontroller into an analog signal to control the current source,
   the analog-to-digital converters (22, 23) which convert the analog signal into the digital signal, which will be recognized by the microcontroller, and
   the resistor (24) which is used for converting current into voltage.

All components of the electrochemical detector and processor (3) are electrically connected and installed on the substrate. The number of electrochemical circuits in the electrochemical detector and processor (3) can be installed corresponding to the number of the electrochemical sensor (2) on the sensor device.

The housing (4) of the sensor device comprises an area for installing the colorimetric sensor (1), electrochemical sensor (2), and electrochemical detector and processor (3). The housing (4) is in a form that allows an attachment to human body, so the colorimetric sensor (1) and electrochemical sensor (2) contact with the secretion directly and continuously during wearing the sensor device. The housing (4) can be made of a material that is selected from a group consisting of textile, paper, polymer, metal, ceramic and a combination thereof.

Preparation of the Non-Invasive Wearable Sensor Device of this Invention

The Preparation of the Sensor Device can be Carried Out by the Following Steps.

a. Preparation of the Colorimetric Sensor (1)

The base material was cut to an appropriate size. Then, the liquid absorber was prepared as a solution with a concentration of 0.001-10% w/v. The solvent for the solution can be selected from water or acid solution, such as acetic acid, hydrochloric acid, and citric acid. After that, the base material was coated with the liquid absorber solution using a technique selected from immersion or dropping, and then left to dry at a room temperature. A multi-layer coating can be made by using the same or different coating material.

The enzyme specific for target biomarkers was prepared as a solution with a concentration of 1-1,000 units/mL. The enzyme was then immobilized on the base material using a technique selected from dropping, immersion or coating, and then left to dry at 20-30° C. for 5-60 min.

The colorimetric agent was prepared as a solution with a concentration of 0.001 to 10% w/v. The colorimetric agent was then used to coat the base material having the immobilized enzyme obtained from the above step. The coating technique can be selected from dropping, immersion or coating, and then left to dry at 20-30° C. for 5-60 min. Finally, the colorimetric sensor was installed on the substrate to absorb the secretion while the device is being worn.

b. Preparation of the Electrochemical Sensor (2)

Starting with a preparation of the working electrode (7), the base material was cut to an appropriate size, and then coated with the conductive material, which can be in various forms such as solid, liquid, suspension or solution. The concentration of the conductive suspension or solution is in a range of 20-70% w/w. The dispersant or solvent can be selected from water, organic solvent or a mixture of organic solvent. The conductive suspension or solution can additionally contain a mediator. The coating technique can be selected from dropping, immersion, coating or plating. The conductive suspension or solution was then dried out at 20-30° C. for 5-60 min. A multi-step coating can be carried out several times, typically 1-10 times. Then, the solution of liquid absorber was prepared with a concentration of 0.001-10% w/v by using water as a solvent. The base material was then coated with the liquid absorber solution using a technique selected from dropping, immersion, coating, and then left to dry at 20-30° C. for 5-60 min. The coating step using the liquid absorber solution can be done before and/or after the coating step using the conductive material. The multi-step coating can be carried out by using the same or different material. Then, the working electrode (7) prepared from the above explanation, reference electrode (6) and counter electrode (8) were installed on the substrate. Importantly, these three electrodes must contact with the secretion while the device is being worn, but each electrode must not contact with each other. Then, the end of three electrodes (9) were coated with the conductive material with a concentration of 20-70% w/w. The working electrode was immobilized with the enzyme with the concentration of 1-1000 unit/mL using a technique selected from dropping, immersion, coating, and then left to dry at 20-30° C. for 5-60 min.

c. Preparation of the Electrochemical Detector and Processor (3)

The electrochemical detector and processor (3) can be fabricated by connecting the circuit and assembling its components. The components comprise the microcontroller, real-time clock, button, display, battery and electrochemical circuit. The electrochemical circuit comprises the operational amplifier, current source controller, current-to-voltage converter, digital-to-analog converter, analog-to-digital converter and resistor. All components were installed on a substrate selected from textile, paper, polymer, metal, ceramic or a combination thereof. The components were connected electrically. The number of electrochemical circuits in the electrochemical detector and processor (3) which is installed corresponds to the number of electrochemical sensors on the device.

FIG. 4 shows a diagram of components of the electrochemical detector and processor (3) for the sensor device that can detect two biomarkers. The diagram contains a list of duplicated modules including the two electrochemical circuits (11, 25) that are connected to the two electrochemical sensors (17, 26) to specifically detect biomarkers A and B, respectively. Each circuit measures the electrical current flowing through different types of electrical sensor. The workflow of the electrochemical circuit that is used to measure biomarker A can be explained as follows.

Upon connection of the electrochemical sensor (17) to the electrochemical circuit (11), the operational amplifier (18) measures the different voltage of the working electrode (WE) and reference electrode (RE). The different voltage is fed to the microcontroller (12) through the analog-to-digital converter (22). The different voltage is also fed back to the current source controller (19) through the negative input. The current source controller (19) measures the different voltage between the target voltage and the input voltage. The target voltage is determined by the microcontroller (12) through the digital-to-analog converter (21). The measured different voltage of the operational amplifier (18) affects the amount of electrical current fed to the sensor cell through the counter electrode (CE). For the WE and RE to have a determined voltage, a certain level of electrical current must be applied to the CE node. The current-to-voltage converter (20) has functions as follows.

Since the input impedance of the operational amplifier is very high, the electrical current from the sensor cell flows only through WE node across a resistor (24). This shows that the current-to-voltage converter (20) obtains voltage Vc at the analog-to-digital converter (23). The digital signal is fed into the microcontroller so that the current flowing into the sensor cell can be calculated using the equation I=Vc/Rm. The system also includes the battery (14) that is a power source which can be a disposable or rechargeable one. The system also includes the button (15) for changing modes and start the operation in each mode. The system also includes the display (16) for showing the measurement results and the real-time clock module (13) for a current timing signal.

d. Assembling of the Components on the Housing (4)

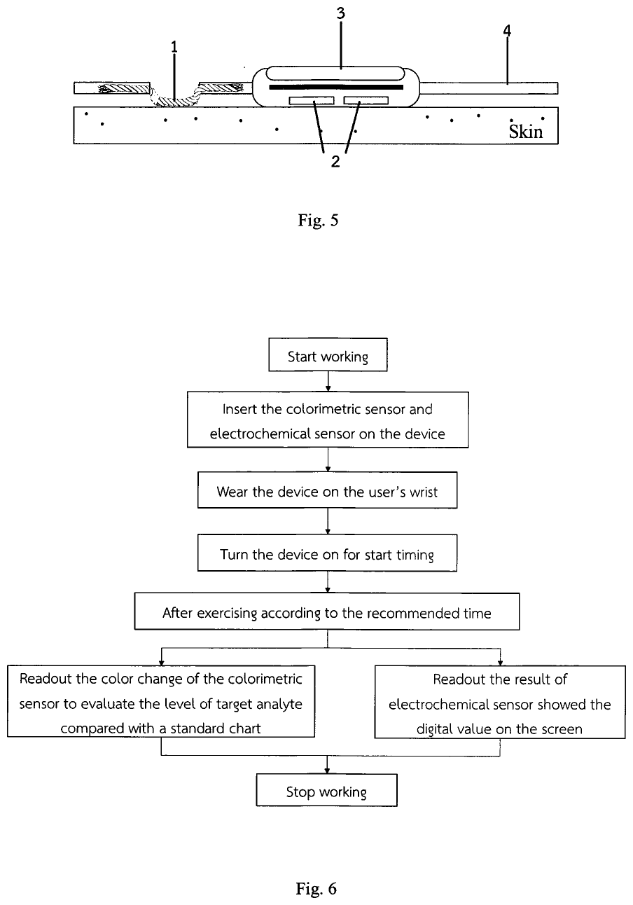

The assembling can be carried out by installing the colorimetric sensor (1), electrochemical sensor (2), and electrochemical detector and processor (3) on the housing (4), which can be selected from textile, paper, polymer, metal, ceramic or a combination thereof. FIG. 5 illustrates a side view of the sensor device and its components being placed on the user's skin for detecting biomarkers in secretion. The colorimetric sensor (1) and electrochemical sensor (2) should be installed such that it enables a continuous absorption of the sample secretion from user's body and easy insertion and removal of the sensor from the sensor device by the user. Furthermore, the user can conveniently interpret the change of color or read the result on the display while wearing the sensor device. One or more of the colorimetric sensor (1) and electrochemical sensor (2) can be installed. All parts of the sensor, detector and electrochemical processor are electrically connected and completely assembled.

FIG. 6 is a flow chart depicting the protocol for using the non-invasive wearable sensor device of this invention for detecting biomarkers in secretion. The sensor device can be used by putting it on user. Some parts of the colorimetric sensor (1) and electrochemical sensor (2) must contact user's skin, so the secretion sample can be absorbed by those sensors. Once the sensor device being worn, the user can observe the color change on the colorimetric sensor (1) and compare it to the standard color chart. For the electrochemical sensor (2), the user can press the button and read the result on the display of the sensor device after the detection process.

The mechanism of the sensor device of this invention is such that when the secretion sample directly contacts with the colorimetric agent, the immobilized specific enzyme will react with the target biomarker and decompose such biomarker. One of the products from the reaction is hydrogen peroxide, which reacts with the colorimetric agent, resulting in the change of color on the colorimetric sensor (1). As for the electrochemical sensor (2), as soon as the secretion sample contacts with the three electrodes, the specific enzyme immobilized on the working electrode (7) will decompose the target biomarker. One of the products from the reaction is hydrogen peroxide, which is a key compound for the electron transfer reaction. After the electron transfer reaction, there is an electrical current in the sensor system which can be detected by the electrochemical technique. The electrochemical detector and processor (3) will measure the current of the counter electrode (8) of the electrochemical sensor (2) on the condition that the voltage of the working electrode (7) and reference electrode (6) is constant. Thus, the current in the system varies directly with the concentration of the target biomarkers.

Exemplary embodiments of the sensor device of this invention include but not limit to the below examples.

Example 1: Certain exemplary techniques and fabrication processes of a wristwatch-based wearable sensor device for simultaneous detection of glucose and lactate in sweat are described below.

Step 1: Preparation of the Colorimetric Sensor (1)
A 0.7% w/v cellulose nanofiber solution was dispersed under ultrasonication for 2 hrs. Then, a 20 cm undyed cotton thread was immersed into the solution under ultrasonication for 1 hr and left to dry at a room temperature for 30 min.
A graphene oxide-chitosan solution was prepared starting from dispersing 20 μL, of 60 mg/mL graphene oxide in 10 mL of acetic acid solution under ultrasonication for 30 min. After that, 0.1 g of chitosan was added to the above solution and homogeneously stirred.
The Graphene oxide-chitosan solution was used for coating the cellulose nanofiber-coated thread by immersing the thread into the graphene oxide-chitosan solution for 30 min, and then left to dry at a room temperature for 30 min.
The thread coated with the cellulose nanofiber/graphene oxide-chitosan was further modified in order to detect different types of biomarker in the following steps.
Preparation of Colorimetric Sensor for Glucose Detection
   To prepare a glucose-specific enzyme solution, 30 unit/mL horseradish peroxidase and 120 unit/mL glucose oxidase were mixed together in 0.1 M phosphate-buffered saline at pH 7.4.
   4 μL, of the glucose-specific enzyme solution were dropped onto the thread coated with the cellulose nanofiber/graphene oxide-chitosan, and then left to dry at room temperature for 15 min.
   To prepare a colorimetric agent solution, 0.6 M potassium iodide solution was diluted in 0.1 M phosphate-buffered saline at pH 7.4.
   4 μL of the colorimetric agent were dropped onto the area immobilized by the glucose-specific enzyme, and then left to dry at room temperature for 15 min.
Preparation of Colorimetric Sensor for Lactate Detection
   To prepare a lactate-specific enzyme solution, 139 unit/mL horseradish peroxidase and 25 unit/mL lactate oxidase were mixed together in 0.1 M phosphate-buffered saline at pH 7.4.
   4 μL of the lactate-specific enzyme solution were dropped onto the thread coated with the cellulose nanofiber/graphene oxide-chitosan, and then left to dry at room temperature for 15 min
   To prepare a colorimetric agent solution, 50 mM 4-aminoantipyrine and 10 mM N-ethyl-N-(2-hydroxy-3-sulfopropyl)-3-methylaniline were mixed together in 0.1 M phosphate-buffered saline at pH 7.4.
   4 μL of the colorimetric agent were dropped onto the area immobilized by the lactate-specific enzyme, and then left to dry at room temperature for 15 min.
   The colorimetric sensor was installed on a flexible substrate on an appropriate position, so the sensor can contact with human body directly.

Step 2: Preparation of the Electrochemical Sensor (2)
A thread cut into a suitable length was coated with a 0.7% w/v cellulose nanofiber solution by immersing the thread into the solution under ultrasonication for 1 hr, and then left to dry at room temperature.
A conductive ink was prepared by dispersing 0.3 mg Prussian blue in 1 mL of carbon nanotube ink solution.
The thread was coated with the carbon nanotube/Prussian blue solution, and then left to dry at room temperature.
The thread coated with the carbon nanotube/Prussian blue was further coated with 0.1% wt chitosan solution, and then left to dry at room temperature.

The thread coated with the carbon nanotube/Prussian blue/chitosan was used as the working electrode, which is ready for further specific modification for the target biomarker.

Preparation of the Counter Electrode (CE) (8) and Reference Electrode (RE) (6)
A flexible PVC sheet was cut to a size of 3×2 cm. Then, the carbon ink was screen-printed on the sheet as a counter electrode, and then dried out in the 55° C. oven for 30 min.
After that, the Ag/AgCl ink was printed on the screen-printed sheet as a reference electrode, and then dried out in the 55° C. oven for 30 min.
The previously prepared working electrode was assembled to the counter electrode and reference electrode to obtain a three-electrode system, wherein each electrode must not contact with each other. Finally, the end of the working electrode was coated with a silver tape.

Preparation of the Working Electrode for Glucose Detection
A glucose-specific enzyme solution was prepared by diluting 120 unit/mL glucose oxidase in 0.1 M phosphate-buffered saline at pH 7.4.
4 μL of glucose-specific enzyme solution were dropped onto the thread coated with the carbon nanotube/Prussian blue/chitosan for using as an electrochemical sensor for glucose detection, and then left to dry at room temperature.

Preparation of the Working Electrode for Lactate Detection
A lactate-specific enzyme solution was prepared by diluting 50 unit/mL lactate oxidase in 0.1 M phosphate-buffered saline at pH 7.4.
4 μL of lactate-specific enzyme solution were dropped onto the thread coated with the carbon nanotube/Prussian blue/chitosan for using as an electrochemical sensor for lactate detection, and then left to dry at room temperature.

Step 3: Preparation of the Electrochemical Detector and Processor (3)

The circuit was connected and the components including a microprocessor, a real-time clock, a control button, a display, a battery and an electrochemical circuit, which is composed of an operational amplifier, a current source controller, a current-to-voltage converter, a digital-to-analog converter, an analog-to-digital converter, and a resistor are installed on a circuit board. Every component was assembled as shown in FIG. 4. Such circuit consists of two electrochemical circuits to connect with two electrochemical sensors for detecting glucose and lactate.

Step 4: Assembling of Each Components on the Housing (4)

A housing (4) was designed as a wristwatch which has channels for insertion of the colorimetric sensor (1), electrochemical sensor (2), and electrochemical detector and processor (3). Each component mentioned above was assembled together.

Example 2: Certain exemplary techniques and fabrication of standard color chart will now be described.

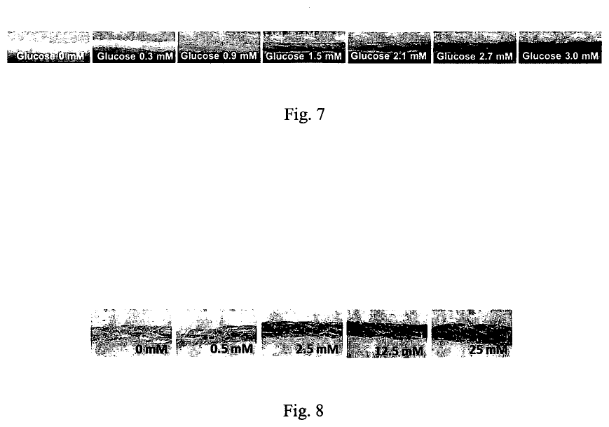

Preparation of Standard Color Chart for Glucose
Various concentrations of the glucose solution were prepared for using as standard glucose solutions. The standard glucose solutions were dropped onto the colorimetric sensor (1), therefore, there are varied color intensities on the colorimetric sensor (1) which are used as a standard color chart. A photo of the color change was taken for further comparison. FIG. 7 demonstrates the standard color chart for glucose which shows the color grading from light yellow to dark brown. The color intensity depends on the concentration of glucose, which means that higher concentration of glucose causes darker shade of color.

Preparation of Standard Color Chart for Lactate
Various concentration of the lactate solution was prepared for using as the standard lactate solutions. The standard lactate solutions were dropped onto the colorimetric sensor, therefore, there are varied color intensities on the colorimetric sensor which are used as a standard color chart. A photo of the color change was taken for further comparison. FIG. 8 demonstrates a standard color chart for lactate which shows the color grading from colorless to purple. The color intensity depends on the concentration of lactate, which means that higher concentration of lactate causes darker shade of color.

Example 3: An example of the use of sensor device for simultaneous detection of glucose and lactate will now be described.
A standard mixture of glucose and lactate solution was prepared as artificial sweat in varied concentrations, and then tested on the sensor device. When the artificial sweat contacts with the working area, the immobilized specific enzyme will decompose the target analytes and give gluconolactone or pyruvate and hydrogen peroxide as reaction products. The reaction of hydrogen peroxide with the colorimetric agent causes a change in color on the colorimetric sensor (1). As for the electrochemical sensor (2), when the artificial sweat contacts with the electrodes, the specific enzyme immobilized on the working electrode will decompose the target analytes. The decomposition reaction produces gluconolactone or pyruvate and hydrogen peroxide which are key compounds for the electron transfer reaction. After the electron transfer reaction, there is electrical current in the electrochemical system which can be detected using the electrochemical technique. The electrochemical detector and processor (3) will measure the current as shown in table 1.

TABLE 1

| Mixture of | | | | |
|---|---|---|---|---|
| glucose and | Glucose concentration | | Lactate concentration | |
| lactate solution | (mM) | | (mM) | |
| in artificial | | electro- | | electro- |
| sweat sample | colorimetric | chemical | colorimetric | chemical |
| 0.3 mM glucose and | ≈0.3 | 0.5 | ≈12.5 | 16.8 |
| 12.5 mM lactate | | | | |
| 1.5 mM glucose and | ≈1.5 | 1.3 | >12.5 | 34.1 |
| 25 mM lactate | | | | |
| 3.0 mM glucose and | ≈3.0 | 2.5 | >12.5 | 53.3 |
| 50 mM lactate | | | | |

### BEST MODE OF THE INVENTION

Best mode of the invention is as described in the detailed description of the invention.

1. A non-invasive wearable sensor device for detecting biomarkers in secretion, the sensor device comprising:
a colorimetric sensor (1) comprising a base material coated with a liquid absorber, a colorimetric reagent and enzyme specific for target biomarkers,
wherein when the colorimetric sensor (1) contacts with the secretion, the enzyme specific for target biomarkers together with the colorimetric reagent causing the color change which is proportional to concentrations of the target biomarkers, and
the colorimetric sensor (1) is installed on a substrate (5) such that it can be attached to and detached from the sensor device;
an electrochemical sensor (2) comprising three electrodes, namely, a reference electrode (RE) (6), a working electrode (WE) (7) and a counter electrode (CE) (8) that are installed on a substrate (10) such that they can be attached to and detached from the sensor device,
wherein the electrochemical sensor (2) is connected to an electrochemical detector and processor (3), and
an end of the three electrodes (9) is coated with a conductive material, and
the working electrode (7) comprises a base material which is coated with a conductive material, liquid absorber and enzyme specific for target biomarkers, and optionally a mediator, and
optionally, more than one colorimetric sensor (1) or electrochemical sensor (2) is installed on the sensor device in order to detect several biomarkers simultaneously, and
when the secretion contacts with the electrochemical sensor (2), the enzyme specific for target biomarkers being on the working electrode (7) reacts with the target biomarkers causing a number of electrons on a surface of the working electrode (7) that are converted into current signals passing through the electrochemical detector and processor (3), the current signals being proportional to concentrations of the target biomarkers, and
the electrochemical detector and processor (3) that works together with the electrochemical sensor (2) comprising:
a microcontroller (12) which serves to control a digital-to-analog converter (DAC) to operate the current source, read the voltage input from a feedback voltage measuring module, read the voltage from a current-to-voltage converter, send the measurable value to a display, monitor and control a working process, and then read a real-time clock signal;
a real-time clock module (13) which serves to generate a current clock signal, and provide the microcontroller (12) with said current clock signal;
a battery (14) as a power source;
a button (15) which is used to switch modes and start the operation;
a display (16) that shows the measured result in the measure mode and shows current clock data;
electrochemical circuits (11, 25) comprising:
an operational amplifier (18) which measures differential voltage between the working electrode (7) and reference electrode (6),
a current source controller (19) which measures differential voltage between its two inputs,
a current-to-voltage converter (20) which converts a current input into a voltage,
a digital-to-analog converter (21) which converts the digital signal from the microcontroller (12) into an analog signal to control the current source,
analog-to-digital converters (22, 23) which convert the analog signal into the digital signal, which will be recognized by the microcontroller, and
a resistor (24) which is used for converting current into voltage,
wherein all components of the electrochemical detector and processor (3) are electrically connected and installed on a substrate, and
a number of the electrochemical circuits (11, 25) installed in the electrochemical detector and processor (3) corresponds to a number of the electrochemical sensor (2) installed on the sensor device;
a housing (4) to which the colorimetric sensor (1), electrochemical sensor (2), and electrochemical detector and processor (3) are installed,
wherein the housing (4) is formed such that allows the colorimetric sensor (1) and electrochemical sensor (2) to contact with the secretion directly and continuously during wearing of the sensor device, and
the housing (4) is made of a material that is selected from a group consisting of textile, paper, polymer, metal, ceramic and a combination thereof.

2. The non-invasive wearable sensor device of claim 1, wherein the base material is made of a textile which is natural fiber, synthetic fiber, conductive fiber or a combination thereof, and is in a form of fiber, thread, fabric or a combination thereof.

3. The non-invasive wearable sensor device of claim 1, wherein the base material is made of paper, polymer, metal, ceramic or a combination thereof.

4. The non-invasive wearable sensor device of claim 1, wherein the base material for the colorimetric sensor (1) and electrochemical sensor (2) are made of the same or different material.

5. The non-invasive wearable sensor device of claim 1, wherein the mediator is selected from a group consisting of metal hexacyanoferrate, Prussian blue, cobalt hexacyanoferrate, cobalt phthalocyanine (CoPc), tetracyanoquinodimethane (TCNQ), potassium ferricyanide, ferrocene and its derivatives and a combination thereof.

6. The non-invasive wearable sensor device of claim 1, wherein the mediator has a concentration in a range of 0.001-10% by weight of the base material.

7. The non-invasive wearable sensor device of claim 1, wherein the liquid absorber is selected from a group consisting of positive ion, negative ion, carbon nanomaterial which is graphene or its derivatives, carbon nanotube, cationic or anionic polymer which is chitosan or its derivatives, cellulose or its derivatives, alginate or its derivatives, pullulan or its derivatives and a combination thereof.

8. The non-invasive wearable sensor device of claim 1, wherein the liquid absorber coated on the colorimetric sensor (1) and electrochemical sensor (2) has a concentration in a range of 0.001-10% by weight of the base material.

9. The non-invasive wearable sensor device of claim 1, wherein the liquid absorber coated on the colorimetric sensor (1) and electrochemical sensor (2) is the same or different material.

10. The non-invasive wearable sensor device of claim 1, wherein the colorimetric reagent is selected from a group consisting of aniline derivatives, i.e. N-ethyl-N-(3-sulfopropyl)-3-methoxyaniline, sodium salt, monohydrate (ADPS), N-ethyl-N-(3-sulfopropyl)aniline, sodium salt (ALPS), N-ethyl-N-(2-hydroxy-3-sulfopropyl)-3,5-dimethoxyaniline, sodium salt (DAOS), N-(2-hydroxy-3-sulfopropyl)-3,5-dimethoxyaniline, sodium salt (HDAOS), N,N-bis(4-sulfobutyl)-3,5-dimethylaniline, disodium salt (MADB), N-ethyl-N-(2-hydroxy-3-sulfopropyl)-3,5-dimethylaniline, sodium salt, monohydrate (MAOS), N,N-bis(4-sulfobutyl)-3-methylaniline, disodium salt (TODB), N-ethyl-N-(2-hydroxy-3-sulfopropyl)-3-methylaniline, sodium salt, dihydrate (TOOS), N-ethyl-N-(3-sulfopropyl)-3-methylaniline, sodium salt, monohydrate (TOPS); benzidine derivatives, i.e. 3,3′-,5,5′-tetramethylbenzidine (TMBZ), 3,3′-,5,5′-tetramethylbenzidine, dihydrochloride, dihydrate, (TMB-HCl), 3,3-diaminobenzidine, tetrahydrochloride (DAB), 4-aminoantipyrine, potassium iodide; azo dyes; triphenylmethane dyes; fluorescent dyes; acridine dyes; miscellaneous dyes; anthraquinone dyes; sulfonephthalein dyes; benzein dyes; xanthene dyes; phthalein dyes; thiazole dyes; coumarin dyes; chalcone dyes; nitro dyes; heterocyclic dyes; polymethine dyes; flavone dyes; indigoid dyes; naphthalene dyes; azine dyes; oxazine dyes; hydrazide dyes; quinoline dyes; styryl dyes; oxazone dyes, i.e. bromocresol green, bromophenol red, methyl orange, methyl red, phenolphthalein, thymol blue, litmus, phenol red and a combination thereof.

11. The non-invasive wearable sensor device of claim 1, wherein the colorimetric reagent has a concentration in a range of 0.0001-10% by weight of the base material.

12. The non-invasive wearable sensor device of claim 1, wherein the enzyme specific for target biomarkers is selected from a group consisting of oxidase enzymes, i.e. glucose oxidase, horseradish peroxidase, lactate oxidase, cholesterol oxidase, creatinine amidohydrolase, urease and a combination thereof.

13. The non-invasive wearable sensor device of claim 1, wherein the enzyme specific for target biomarkers coated on the colorimetric sensor (1) and electrochemical sensor (2) has a concentration in a range of 0.01-1,000 units per gram of the base material.

14. The non-invasive wearable sensor device of claim 1, wherein the enzyme specific for target biomarkers coated on the colorimetric sensor (1) and electrochemical sensor (2) is the same or different enzyme.

15. The non-invasive wearable sensor device of claim 1, wherein the reference electrode (6) is an ink or electrode which comprises carbon or Ag/AgCl as a main component.

16. The non-invasive wearable sensor device of claim 1, wherein the counter electrode (8) is an ink or electrode which comprises carbon, Ag/AgCl or platinum (Pt) as a main component.

17. The non-invasive wearable sensor device of claim 1, wherein the conductive material is selected from a group consisting of carbon-based nanomaterials, i.e. graphene or its derivatives, carbon nanotubes; metal-based nanoparticles, i.e. gold, silver, platinum, nickel, copper; conductive polymer, i.e. polyaniline, polypyrrole, poly(3,4-ethylenedioxy thiophene): polystyrene sulfonate; conductive ink or adhesive, i.e. Ag/AgCl ink, carbon ink; conductive tape, i.e. silver tape, copper tape and a combination thereof.

18. The non-invasive wearable sensor device of claim 1, wherein the conductive material coated on the working electrode (7) and the end of the three electrodes (9) has a concentration in a range of 1-1000% by weight of the base material.

19. The non-invasive wearable sensor device of claim 1, wherein the conductive material coated on the working electrode (7) and the end of the three electrodes (9) is the same or different material.

20. The non-invasive wearable sensor device of claim 1, wherein the substrate of the colorimetric sensor (1), electrochemical sensor (2) and electrochemical detector and processor (3) is selected from a group consisting of textile, paper, polymer, metal, ceramic and a combination thereof.

21. The non-invasive wearable sensor device of claim 1, wherein the substrate of the colorimetric sensor (1), electrochemical sensor (2) and electrochemical detector and processor (3) is the same or different material.
